(19) United States
(12) Patent Application Publication
(10) Pub. No.: US 2020/0397317 A1
(43) Pub. Date: Dec. 24, 2020

(54) VISUALIZATION OF 4D DYNAMIC PULSATILE FLOW

(71) Applicant: CEDARS-SINAI MEDICAL CENTER, Los Angeles, CA (US)

(72) Inventors: Moise Danielpour, Los Angeles, CA (US); Nader Binesh, West Hills, CA (US); Barry Pressman, Beverly Hills, CA (US); Marcel Maya, Los Angeles, CA (US); Nestor Gonzalez, Los Angeles, CA (US)

(73) Assignee: CEDARS-SINAI MEDICAL CENTER, Los Angeles, CA (US)

(21) Appl. No.: 16/975,203

(22) PCT Filed: Mar. 20, 2019
(86) PCT No.: PCT/US2019/023190

§ 371 Date: Aug. 24, 2020

Related U.S. Application Data
(60) Provisional application No. 62/647,178, filed on Mar. 23, 2018.

(51) Int. Cl. A61B 5/026; A61B 5/055; A61B 5/00; A61B 5/021; A61B 5/0285; A61B 5/02; G01R 33/561; G01R 33/563; G01R 33/385; G01R 33/48

(52) U.S. Cl. CPC A61B 5/0263; A61B 5/055; A61B 5/0042; A61B 5/4842; A61B 5/02108; A61B 5/0285; G01R 33/4822; A61B 5/02007; A61B 5/4088; G01R 33/5614; G01R 33/56325; G01R 33/385; A61B 5/02014

(57) ABSTRACT

Systems and methods for monitoring pulsatile flows are disclosed. One method includes obtaining a plurality of magnetic resonance imaging (MRI) scans of a biological structure, acquired consecutively in time, each of the MRI scans acquired using a modified True FISP sequence. The method also includes assembling the plurality of scans into a video file of the biological structure and identifying pulsatile features within the video file.

### CROSS REFERENCE TO RELATED APPLICATIONS

This application claims priority under 35 U.S.C. § 119 to U.S. Provisional Application No. 62/647,178, entitled, “Visualization of 4D Dynamic Pulsatile Flow,” filed Mar. 23, 2018, the contents of which are incorporated herein by reference.

### FIELD OF THE INVENTION

The present invention relates to measurement of fluid flow in biological tissues, and more specifically to apparatus and methods for measurement of dynamic pulsatile flow of fluids in biological tissues.

### BACKGROUND

Numerous homeostatic processes in the brain, such as cerebral blood flow and maintenance of interstitial fluid equilibrium, critically depend on the regulation of intra-cranial pressure (ICP) and fluid flow. The maintenance of adequate blood flow to the brain is critical for normal brain function. Cerebral blood flow, its regulation and the effect of alteration in this flow with disease, has been studied extensively. Conventional systems and methods have identified that cerebral blood flow can be affected in various ways by certain disease.

Cerebral blood flow is not steady, however; the systolic increase in blood pressure over the cardiac cycle causes regular variations in blood flow into and throughout the brain. These regular variations are synchronous with the heartbeat and comprise pulsations in flow and pressure. These pulsations in flow and pressure of the blood in the brain are in turn transmitted to brain parenchyma and all of the fluids contained therein—including cerebrospinal fluid (CSF). Conventional systems and methods look to measurements of pulsatile pressure in the brain as markers for certain diseases. For example, in hydrocephalus and traumatic brain injuries, large changes in intracranial pressure and in the biomechanical properties of the brain can lead to significant changes in pressure and flow pulsatility.

The pulsatile blood flow, which is created by the heart, is transferred to the aorta and its proximal branches, which are rich in elastin that dampens the pulsations. Cerebral arterial pulsatile flow is dampened in a progressive manner when moving from proximal parts to distal parts of the cerebral arterial system. Dampening is more pronounced in younger than older subjects. With aging, and a corresponding increased large artery stiffness, there is an increased pulsatile stress to the brain microvasculature and other vascular pathology. Pulsatile stress to highly-perfused organs, such as the brain, is considered to be (1) a possible cause of microvascular damage and (2) a contributor to the progression of pathological states, such as dissection or aneurysmal disease. This pulsatile stress can additionally be involved in the pathophysiology of lacunar infarcts, normal pressure hydrocephalus, mild cognitive impairment, and dementia.

A common method to investigate aortic stiffness is aortic pulse wave velocity (PWV), which can help predict cardiovascular morbidity and mortality. Flow pulsations in cerebral arteries can be investigated using Doppler ultrasonography and phase-contrast magnetic resonance imaging. Although Doppler ultrasonography is widely available, it has several drawbacks; most importantly, investigations are limited to the proximal branches of the cerebral arterial tree and cannot predict impact to distal branches of the arterial tree, or any other locations in the human body. Therefore, in particular, conventional systems and methods have not used Doppler ultrasonography and phase-contrast magnetic resonance imaging to effectively quantify many aspects of intracranial pulsatility.

Three primary techniques have been used in conventional systems to quantify aspects of intracranial pulsatility (ICP): (1) continuous ICP monitoring, (2) transcranial Doppler ultrasound (TCD), and (3) magnetic resonance imaging (MRI). ICP monitoring, which requires invasive provedures, can measure pressure pulsatility. ICP monitoring requires placement of a pressure sensor within the brain, either in the parenchyma, ventricle, epidural space, or spinal CSF space. TCD and MRI provide measures of flow pulsatility and are non-invasive: TCD measures the velocity of blood flow in the large arteries using a transducer placed against the skull, while MRI measures the net flow waveform over the cardiac cycle, within the large intracranial arteries or veins or within well-defined CSF pathways (e.g., the cerebral aqueduct or at the cra-niocervical junction (CCJ)). Thus, ICP is a pressure-based measure of pulsatility, while TCD and MRI are flow-based. Comparing pulsatility measures across modalities (for example, between ICP, TCD, and MRI methods) does not always yield valid results, because the methods are not equivalent and assess different aspects of cardiac pulsatility.

Therefore, systems and methods are needed for accurately, and non-invasively, measuring ICP.

### SUMMARY

A first embodiment of the present disclosure provides for a method of obtaining and analyzing MRI scans. The method can provide for first obtaining a plurality of magnetic resonance imaging (MRI) scans of a cerebral vessel. Each of the plurality of MRI scans can include a modified True FISP sequence. The modified True FISP sequence can include modified True FISP parameters related to a resolution and a speed of imaging. The modified True FISP sequence can yield a scan image with identifiable physiological structures. The method can then provide for outputting scan images based on the plurality of MRI scans. Based on the scan images of the plurality of MRI scans, the method can provide for identifying a flow within the cerebral vessel. Based on the identified flow within the cerebral vessel, the method can provide for identifying pulsatile features of the identified flow. The method can then provide for determining whether the identified pulsatile features are related to a disease state. The method can provide for outputting an indication fo the disease state, based on determining whether the identified pulsatile features are related to a disease state.

In some examples, the modified True FISP parameters can include field of view and/or resolution matrix.

In some examples, the plurality of MRI scans can be obtained consecutively over a set period of time. Between each scan in the plurality of MRI scans, there can be a set interval. For example, the set interval can be one second, two seconds, three seconds, four seconds, five seconds, or any other period of time as known in the art.

In some examples, visualizing the flow within the cerebral vessel can include generating 3-D representation of fluid moving through the cerebral vessel over a set period of time. Such a visualization can therefore be a four-dimensional, dynamic visualization of the pulsatile flow.

In some examples, visualizing the flow within the cerebral vessel can include assembling the plurality of MRI scans into a video file.

In some examples, the identified pulsatile features can include flow pressure, flow pulsatility, and/or flow velocity.

In some examples, determining whether the identified pulsatile features are related to a disease state can include comparing the identified pulsatile features to corresponding threshold values. The corresponding threshold values can be based on values of pulsatile features for a particular disease state. Comparing the identified pulsatile features to corresponding threshold values can include determining whether the pulsatile features is higher or lower than a threshold value.

In some examples, the disease state can include any of the following: dementia, stroke, hydrocephalus, arteriovenous malformations, aneurysms, renal artery stenosis, development of atherosclerotic disease, or peripheral vascular disease.

A second embodiment of the present disclosure can provide for a system, which includes an MRI system, a computing device, and a memory. The computing device can be communicatively coupled to the MRI system. The memory can have a stored computer program for operating the computing device. The computer program can have a plurality of code sections for causing the computing device to perform a series of steps. The steps can provide for controlling the MRI system so as to acquire a plurality of consecutive MRI scans of a cerebral vessel using a modified True FISP sequence. Each of the plurality of consecutive MRI scans can output a scan image. The steps can provide for identifying a flow within the cerebral vessel based on the scan images of each of the plurality of consecutive MRI scans. The steps can further provide for identifying a feature of the flow related to a disease state. The steps can then provide for outputting an indication of the identified feature to a display.

In some examples, identifying the flow within the cerebral vessel can include assembling the plurality of consecutive MRI scans into a video file.

In some examples, the identified feature of the flow can include any of: a flow pressure, a flow pulsatility, and/or a flow velocity.

In some examples, the modified True FISP sequence can include a modified field of view and/or a resolution matrix.

In some examples, the steps can further provide for assessing a disease state of the cerebral vessel based on the identified feature of the flow. Assessing the disease state can include comparing a value of the identified feature to a threshold value associated with the identified feature.

In some examples, the disease state can include any of the following: dementia, stroke, hydrocephalus, arteriovenous malformations, aneurysms, renal artery stenosis, development of atherosclerotic disease, or peripheral vascular disease.

### BRIEF DESCRIPTION OF THE DRAWINGS

FIG. 1 is a diagram of an exemplary True FISP sequence that is useful for understanding the various embodiments.

FIGS. 2A-2E show an exemplary set of parameters for an unmodified True FISP sequence that is useful for understanding the various embodiments.

FIG. 3 shows an exemplary methodology, according to an embodiment of the present disclosure.

FIGS. 4A-4E show an exemplary set of parameters for a modified True FISP sequence, according to the various embodiments.

FIG. 5 shows sample frames from a video of acquisition using MRI phase contrast and a modified True FISP sequence, according to the various embodiments for a first subject.

FIG. 6 shows sample frames from a video of acquisition using MRI phase contrast and a modified True FISP sequence, according to the various embodiments for a second subject.

FIG. 7 shows sample frames from a video of acquisition using MRI phase contrast and a modified True FISP sequence, according to the various embodiments for a third subject.

FIG. 8 shows sample frames from a video of acquisition using MRI phase contrast and a modified True FISP sequence, according to the various embodiments for a fourth subject.

FIG. 9 shows sample frames from a video of acquisition using MRI phase contrast and a modified True FISP sequence, according to the various embodiments for a fifth subject.

FIG. 10 shows sample frames from a video of acquisition using MRI phase contrast and a modified True FISP sequence, according to the various embodiments for a sixth subject.

FIGS. 11 and 12 show an exemplary MRI system which can be used to implement the various embodiments.

### DETAILED DESCRIPTION

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. In other instances, well-known structures or operations are not shown in detail to avoid obscuring the invention. The present invention is not limited by the illustrated ordering of acts or events, as some acts may occur in different orders and/or concurrently with other acts or events. Furthermore, not all illustrated acts or events are required to implement a methodology in accordance with the present invention.

In view of the limitations of existing methods, the present disclosure is directed to a new methodology for measurement of either CSF or blood flow pulsatility. A methodology according to the present disclosure can (1) provide absolute velocity information, as in a conventional TCD method, and (2) provide image information in a two- or three-dimensional format to allow extraction of net flow measurements, as in conventional MRI methods. Therefore, a methodology, according to the present disclosure, can provide quantitative and qualitative measures of both velocity and flow pulsatility.

In particular, the present disclosure provides a methodology in which blood flow pulsaility is measured in a four-dimensional (4D) fashion using MRI scans. The 4D fashion provides for a three-dimensional flow to be visualized in time over the entire cardiac cycle. Accordingly, the present disclosure can generate MRI imagery that adds three-dimensional visualization of turbulence and pulsatile flow. This visualization can then be used as a method to further delineate changes in disease states when compared with normal pulsatile blood flow. This methodology can therefore be used to assess severity of disease, probability of progression, and success of intervention for various disease states. Exemplary disease states can include, but are not limited to, dementia, stroke, hydrocephalus, arteriovenous malformations, aneurysms, renal artery stenosis, development of atherosclerotic disease, and peripheral vascular disease, among other disease states. The present disclosure contemplates that the methodology described herein can be utilized for diagnosis of a variety of other disease states not listed above.

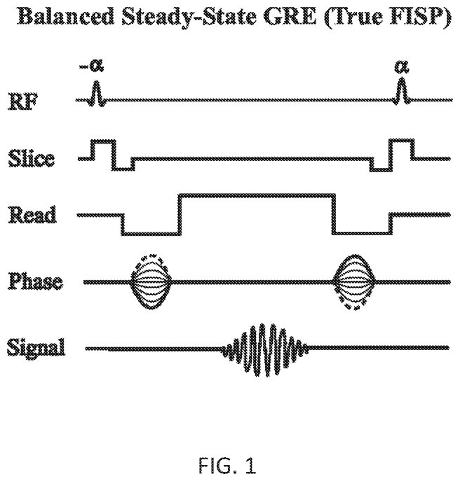

The new methodology involves the use of a new MRI sequence to image pulsatile flow. In particular, the new MRI sequence is a variation of a “trufi” (True FISP) sequence, which is steady-state coherent sequence with balance gradients on all 3 axes. FIG. 1 shows an exemplary True FISP sequence that is useful for understanding the various embodiments.

In a True FISP sequence, as shown in FIG. 1, as the echo is formed right at the center of the interval, the resulting image intensity is proportional to T2/T1. In conventional imaging sequence, gradient echo images are affected by T2*, such that at long TEs, the T2* would be the dominant factor. By contrast to conventional imaging sequences, in a True FISP sequence, T2 is the main contributor and the inhomogeneity is focused by the balanced gradients. The T2/T1 ratio is low for many tissues, except for fat and CSF/fluids. This makes the True FISP sequence excel at displaying fluids in larger chambers and vessels of the heart; hence, it is widely used in cardiac imaging. The images captured from a True FISP sequence can be then played back as a movie (CINE), which is very helpful for visualizing the flow. Thus, True FISP is designed for cardiac scanning, and is designed for that level of flow and speed.

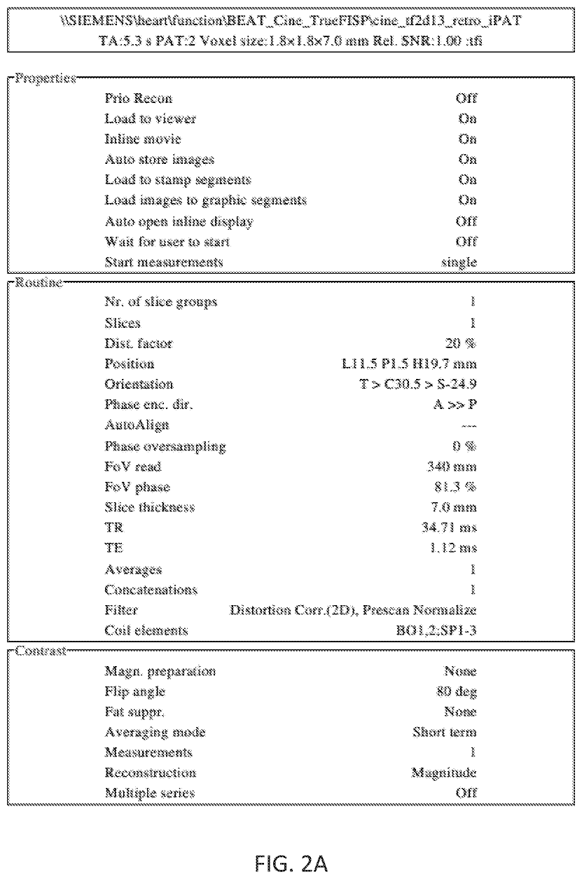

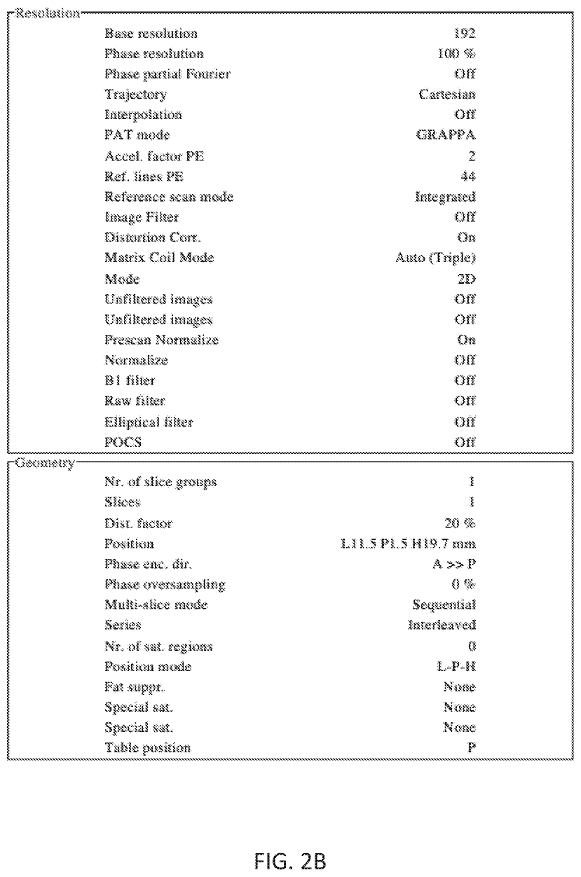

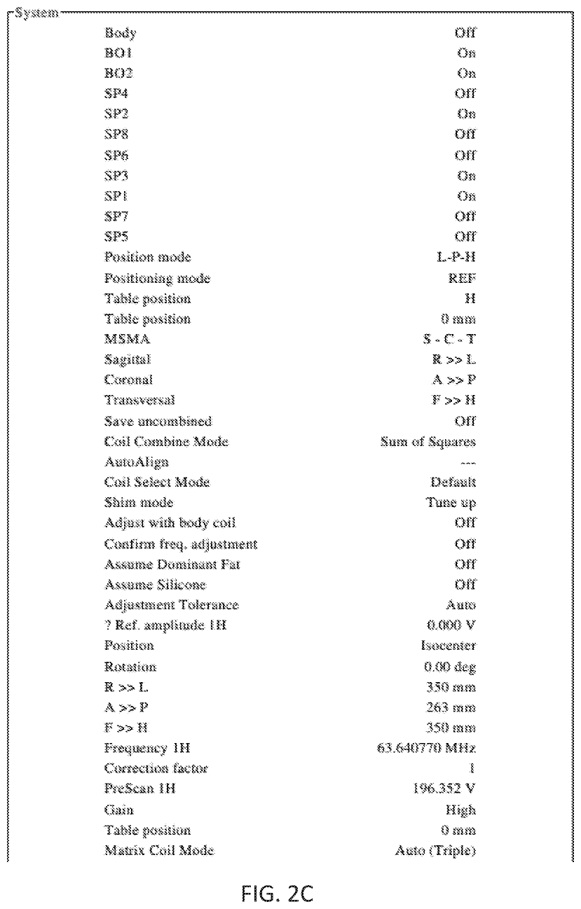

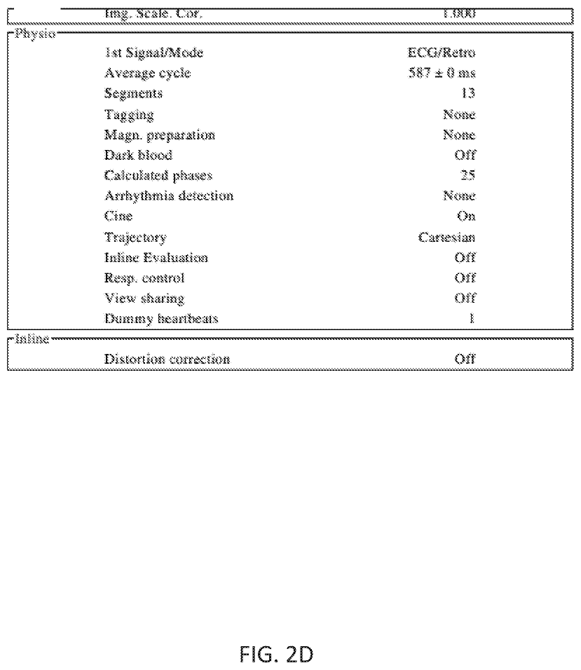

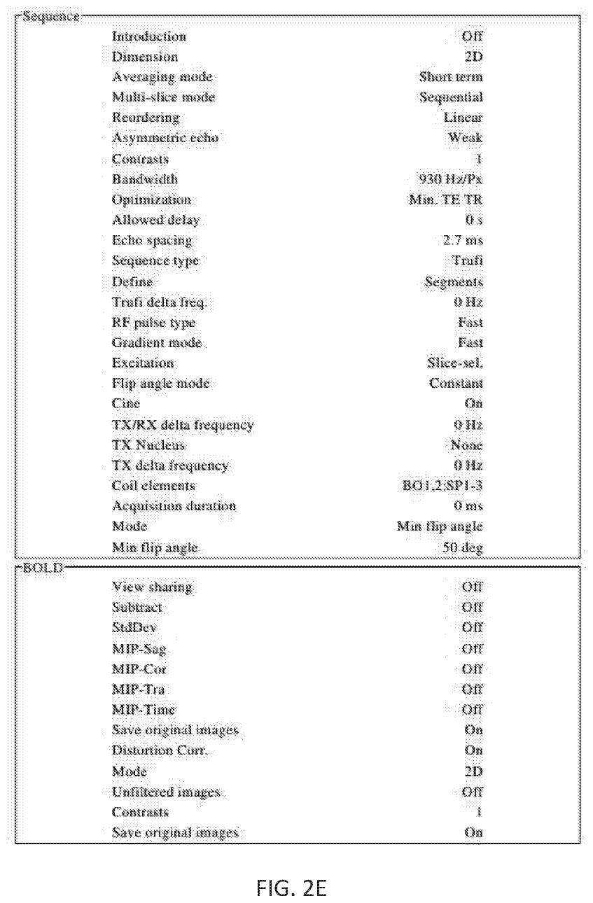

FIGS. 2A-2E show exemplary parameters for a True FISP sequence, as conventionally used. As discussed above with regards to FIG. 1, the parameters shown in FIGS. 2A-2E provide for T2 as the main contributor, with a low T2/T1 ratio in many tissues. An image produced based on the parameters of FIGS. 2A-2E can display fluid movement in large chambers, such as vessels of the heart. However, the conventional parameters shown in FIGS. 2A-2E cannot produce an image showing flow pulsatility in distal branches of the arterial tree or any other locations in the human body (besides the larger chambers and vessels of the heart).

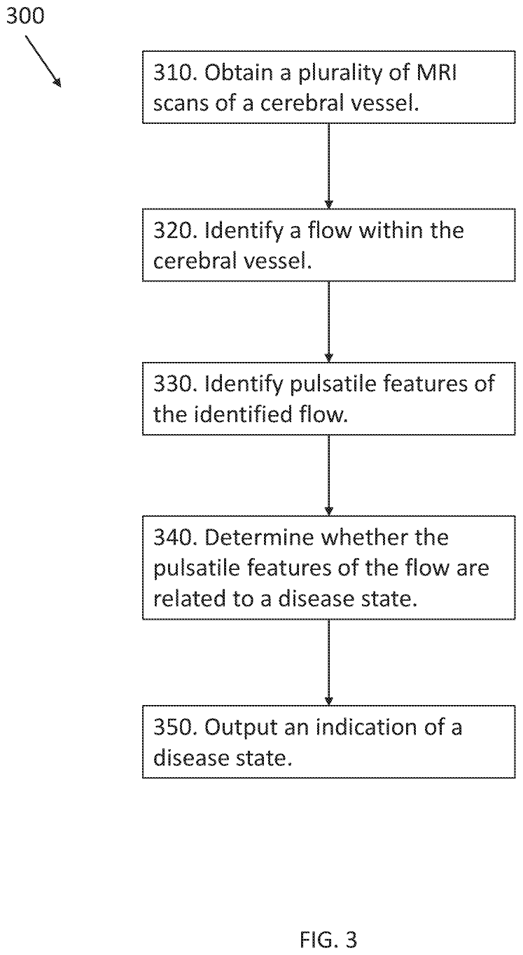

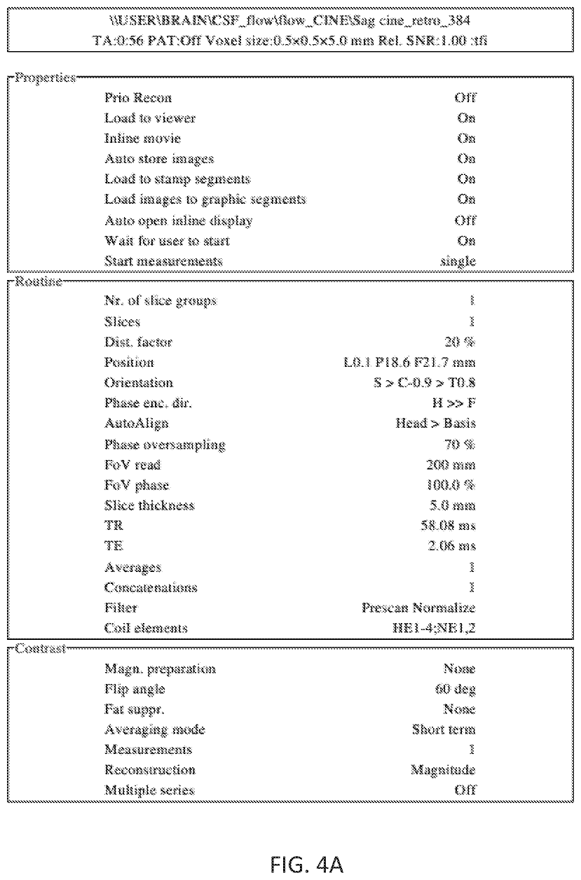

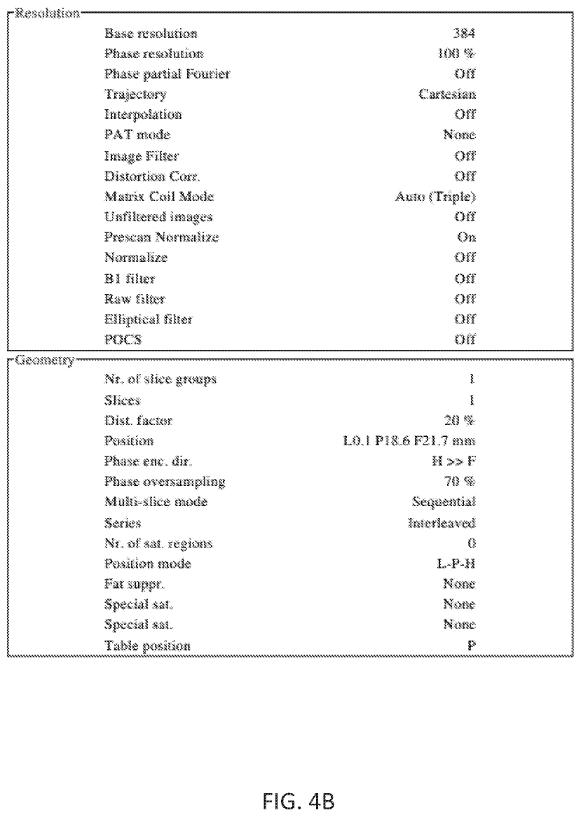

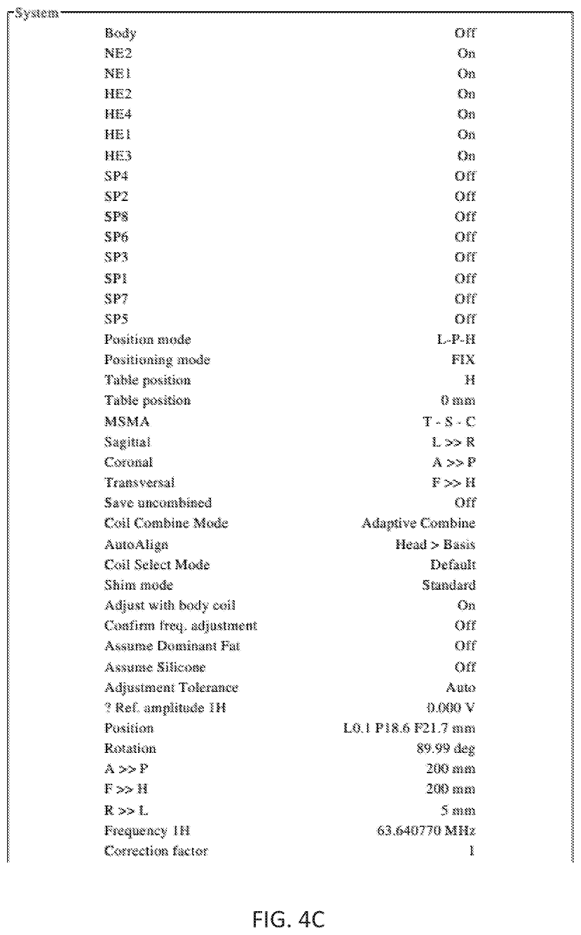

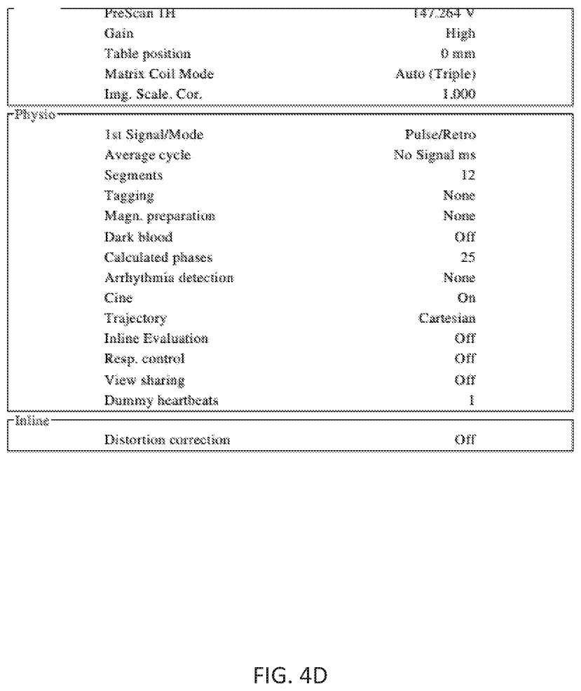

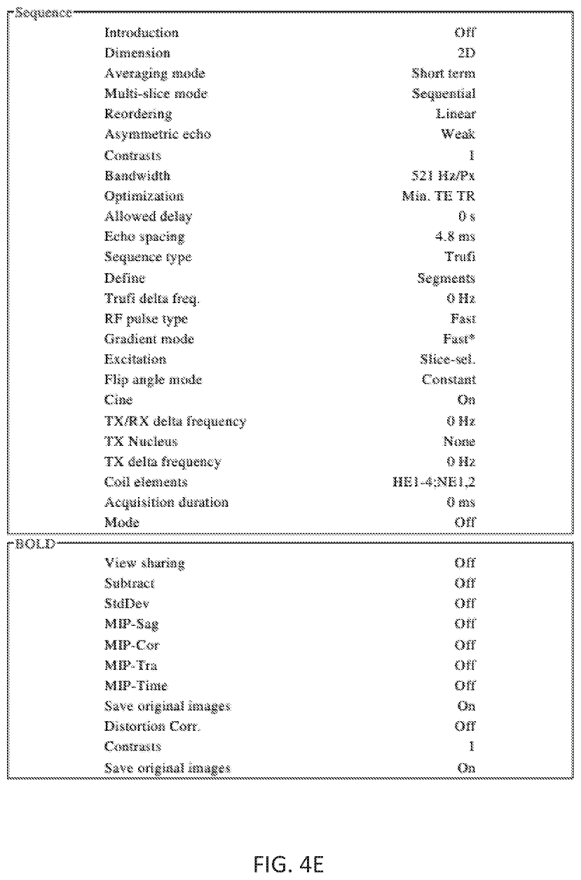

In view of the limitations of existing techniques, the present disclosure provides for a modified True FISP imaging sequence, which surprising allows for a level of sensitivity and precision using time-gated magnetic data to visualize pulsatile flow in a tubular blood vessel. An exemplary methodology is shown in FIG. 3, and an exemplary set of parameters according to the present disclosure is shown in FIGS. 4A-4E. Adapting the conventional True FISP sequence, discussed with respect to FIGS. 1-2E, was especially challenging because tubular blood vessels move (pulsate, expand, and contract) in response to changes in blood pressure. Additionally, the tubular blood vessels have a greater degree of pulsation in relation to their size than larger vessels of the heart, for example. Despite these difficulties of visualizing tubular blood vessels, the disclosed set of parameters (FIGS. 4A-4E) and the disclosed methodology (FIG. 3) modifies the time between pulse, amplitude, and phases to allow for visualization of pulsatile blood flow in tubular vessels (e.g. arterial and venous blood flow) in cerebral vasculature.

The exemplary methodology 300 of FIG. 3 can begin at step 310 by obtaining MRI scans of a cerebral vessel of a patient. Each of the MRI scans can include a modified True FISP sequence (for example, the modified True FISP sequence shown in FIGS. 4A-4E). This modified True FISP sequence can include modified True FISP parameters which are related to a resolution and a speed of imaging. The modified True FISP parameters of step 310 can therefore yield a scan image with identifiable physiological structures. The modified True FISP parameters can include some or all of these parameters: field of view (FoV), base resolution or matrix, slice thickness, repetition time (TR), echo time (TE), flip angles, bandwidth, echo spacing, and gating. For instance, in some examples, modification of some or all of these parameters led to changes in the resolution and speed of imaging surprising allowed for visualization of flow in small, pulsating vessels at the base of the skull.

The changes in these parameters can be significant compared to a conventional True FISP sequence. Referring briefly to FIGS. 4A-4E and FIGS. 2A-2E, Table 1, below, compares selected values from the modified True FISP sequence of the present disclosure (as shown in FIGS. 4A-4E) with the unmodified True FISP parameters (as shown in FIGS. 2A-2E):

TABLE 1
SEQUENCE PARAMETER CHANGES

| Parameter | NEW | | True FISP | | % Change |
|---|---|---|---|---|---|
| FoV | 200 | mm | 340 | mm | −41.2% |
| Matrix | 384 | | 192 | | +100% |
| Slice Thickness | 5 | mm | 7 | mm | −28.6% |
| TR | 52 | ms | 34.71 | ms | +49.8% |
| TE | 2 | ms | 1.12 | ms | +78.6% |
| Flip Angle | 60 | | 80 | | −25% |
| BW | 750 | | 930 | | −19.4% |
| Echo Spacing | 4.2 | ms | 2.7 | ms | +55.6% |

| | | | |
|---|---|---|---|
| Gating | Pulse/Retro/12 | ECG/Retro/13 | N/A |
| | Segments | Segments | |

FIGS. 4A-4E, therefore, show an exemplary set of parameters which can provide for an MRI scan running in about 40 seconds, which produces multiple images of a Sagittal view. Such a scan can demonstrate flowing CSF as well as blood in the Basilar artery at a resolution of 0.5×0.5 cm² for a whole cardiac cycle. As discussed further below with respect to step 320 of FIG. 3, the multiple images can then be compiled as a movie. The resulting movie can show the pulsatile flow as blood rushes through the Basilar artery.

Referring back to step 310 in FIG. 3, the MRI scans can be obtained consecutively over a set period of time, with a set interval between each scan of the plurality of MRI scans. The set interval can be half a second, one second, two seconds, three seconds, five seconds, ten seconds, or any other period of time as readily contemplated by one skilled in the art.

The MRI scans can provide 2-dimensional images of the cerebral tissue of the patient, for example, along an axial plane. The 2-dimensional slices can be joined together to produce a 3-dimensional model of the scanned area.

Methodology 300 can provide for outputting scan images based on the plurality of MRI scans after the plurality of MRI scans are obtained in step 310.

After obtaining the MRI scans, the methodology 300 can then proceed to step 320 to provide for identifying a flow within the cerebral vessel. The flow can be identified based on the scan images of each of the plurality of MRI scans.

In some examples, the flow can be identified by visualizing the flow based on the MRI scans, in either the 2-dimensional slices or the 3-dimensional model. For example, the present disclosure can provide for generating a 3-D representation of fluid moving through the cerebral vessel over a set period of time. The set period of time can be the length of the time that it took to obtain the MRI scans. In some examples, step 320 can provide for assembling MRI scans into a video file.

The methodology 300 can then proceed to step 330 to provide for identifying pulsatile features of the identified flow. The identified pulsatile features can include, for example, flow pressure, flow pulsatility, and flow velocity.

For example, the pulsatile features can be based on a video file generated from the MRI scans in step 320.

The methodology 300 can then proceed to step 340 and provide for determining whether the pulsatile features of the flow are related to a disease state. In some examples, step 340 can provide for comparing an identified pulsatile feature of the scanned cerebral vessel to a threshold value for the pulsatile feature. In some examples, the threshold value can represent a value for the pulsatile feature when the cerebral vessel is in a disease state. In other examples, the threshold value can represent a value for the pulsatile feature when the cerebral vessel is not in a disease state. The disease state can be, for example, dementia, stroke, hydrocephalus, arteriovenous malformations, aneurysms, renal artery stenosis, development of atherosclerotic disease, peripheral vascular disease, or any other disease state which affects pulsatile features in a cerebral vessel.

The methodology 300 can then proceed to step 350 and output an indication of a disease state. In some examples of step 350, the indication can identify whether a patient has a particular disease state and a severity of the disease state. The indication can be output on a display.

Therefore, the methodology 300 of FIG. 3 can provide for the visualization of the CSF, blood flow in arteries, and blood flow veins at the skull base. Altogether, systems and methods, according to the present disclosure, can visualize the flow in sagittal plane close to pons. As a result, the methodology of the present disclosure provides the most accurate depiction of pulsatile blood and CSF flow in the brain. Consequently, thus methodology can be used to identify any issue that could have gone undetected in conventional static imaging or even dynamic imaging using conventional True FISP sequences.

### EXAMPLES

The examples shown here are not intended to limit the various embodiments. Rather they are presented solely for illustrative purposes.

Turning first to FIGS. 2A-2E, there is shown an exemplary set of parameters for a True FISP sequence for a Siemens Avanto scanner (1.5T). In contrast, the exemplary set of parameters for a new sequence in accordance with the present disclosure is shown in FIGS. 4A-4E. Using the parameters of FIGS. 4A-4E, the advantages of the new sequence are readily apparent as compared to conventional MRI methods for monitoring blood and CSF flow. In particular, the parameters of FIGS. 4A-4E provide clear superiority to MRI phase contrast methods.

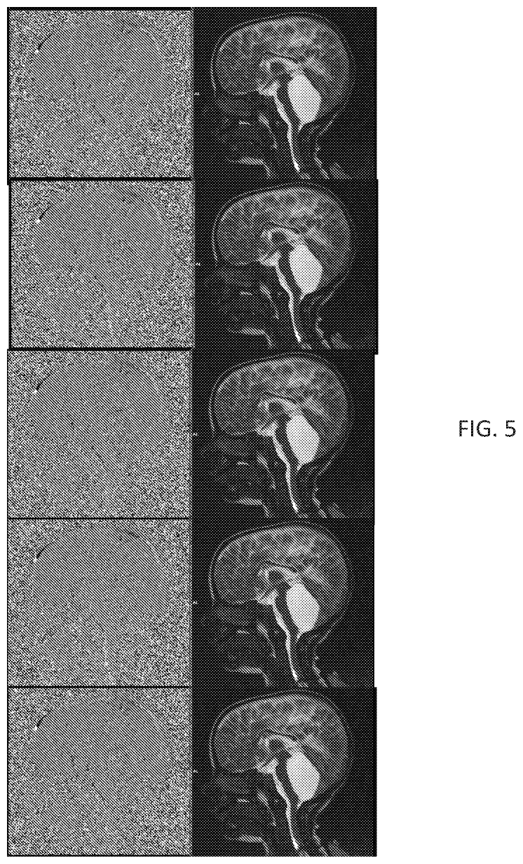

Turning now to FIG. 5, there is shown a series of five two-dimensional (2D) images acquired using a conventional MRI scan with phase contrast (left) and an MRI scan acquired using the new sequence discussed above (right). The subject was a 15-year-old male. In FIG. 5, each of the images represents a sample image acquired every two seconds from a series of 88 consecutive scans. As shown in FIG. 5, the difference between the phase contrast images and the images using the new sequence is readily apparent. In particular, the images on the right (generated with a sequence according to the present disclosure) are significantly clearer, allowing physiological structures to be easily identified than in the images on the left (generated with a conventional sequence as in the prior art). Moreover, when images from all 88 scans are played consecutively as a movie, the pulsating of blood flow and CSF can be observed clearly.

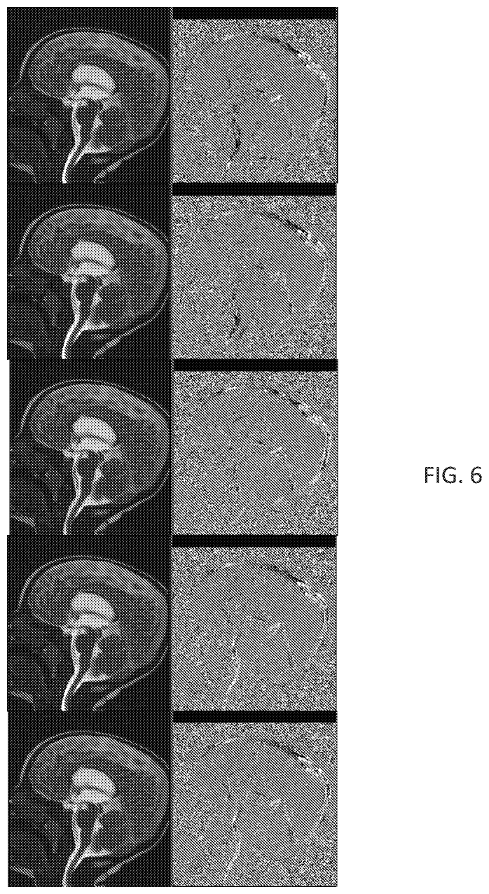

Turning now to FIG. 6, there is shown a series of five 2D images acquired using a conventional MRI scan with phase contrast (right) and an MRI scan acquired using the new sequence discussed above (left). The subject was a 22-year-old male. In FIG. 6, each of the images represents a sample image acquired every two seconds from a series of 150 consecutive scans. As shown in FIG. 6, the difference between the phase contrast images and the images using the new sequence is readily apparent. In particular, the images on the right (generated with a sequence according to the present disclosure) are significantly clearer, allowing physiological structures to be easily identified than in the images on the left (generated with a conventional sequence as in the prior art). Moreover, when images from all 150 scans are played consecutively as a movie, the pulsating of blood flow and CSF can be observed clearly.

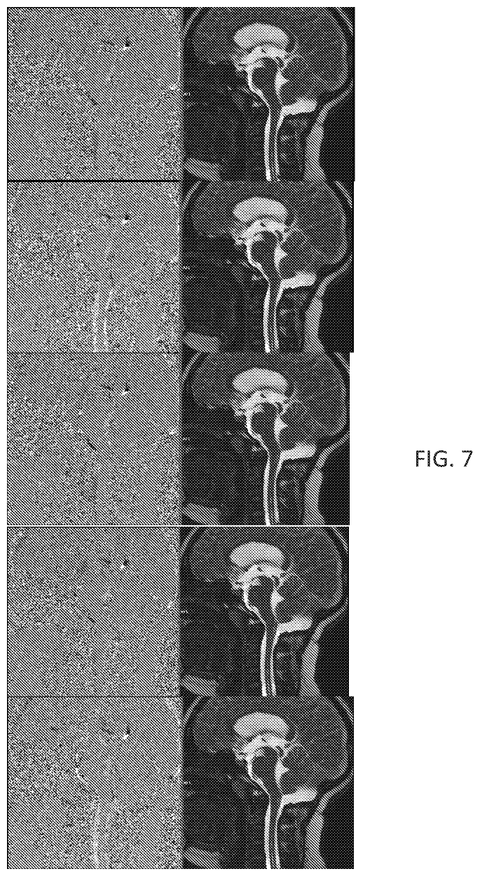

Turning now to FIG. 7, there is shown a series of five 2D images acquired using a conventional MRI scan with phase contrast (left) and an MRI scan acquired using the new sequence discussed above (right). The subject was a 16-year-old female. In FIG. 7, each of the images represents a sample image acquired every two seconds from a series of 108 consecutive scans. As shown in FIG. 7, the difference between the phase contrast images and the images using the new sequence is readily apparent. In particular, the images on the right (generated with a sequence according to the present disclosure) are significantly clearer, allowing physiological structures to be easily identified than in the images on the left (generated with a conventional sequence as in the prior art). Moreover, when images from all 108 scans are played consecutively as a movie, the pulsating for blood flow and CSF can be observed clearly.

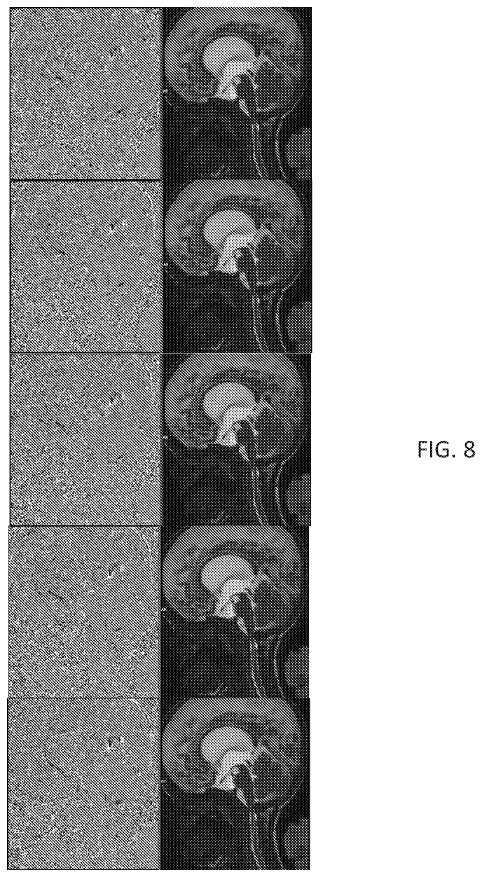

Turning now to FIG. 8, there is shown a series of five 2D images acquired using a conventional MRI scan with phase contrast (left) and an MRI scan acquired using the new sequence discussed above (right). The subject was a 15-year-old male. In FIG. 8, each of the images represents a sample image acquired every two seconds from a series of 112 consecutive scans. As shown in FIG. 8, the difference between the phase contrast images and the images using the new sequence is readily apparent. In particular, the images on the right (generated with a sequence according to the present disclosure) are significantly clearer, allowing physiological structures to be easily identified than in the images on the left (generated with a conventional sequence as in the prior art). Moreover, when images from all 112 scans are played consecutively as a movie, the pulsating for blood flow and CSF can be observed clearly.

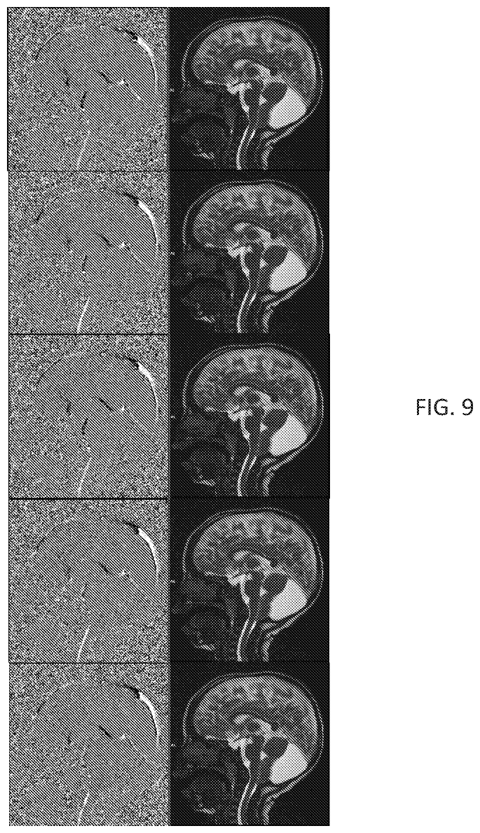

Turning now to FIG. 9, there is shown a series of five 2D images acquired using a conventional MRI scan with phase contrast (right) and an MRI scan acquired using the new sequence discussed above (left). The subject was a 22-year-old male. In FIG. 9, each of the images represents a sample image acquired every two seconds from a series of 117 consecutive scans. As shown in FIG. 9, the difference between the phase contrast images and the images using the new sequence is readily apparent. In particular, the images on the right (generated with a sequence according to the present disclosure) are significantly clearer, allowing physiological structures to be easily identified than in the images on the left (generated with a conventional sequence as in the prior art). Moreover, when images from all 117 scans are played consecutively as a movie, the pulsating for blood flow and CSF can be observed clearly.

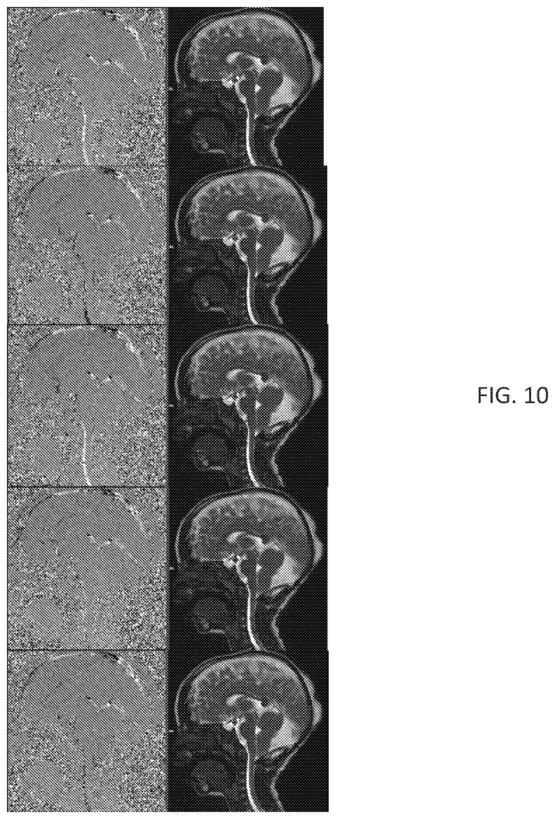

Turning now to FIG. 10, there is show, a series of five 2D images acquired using a conventional MRI scan with phase contrast (right) and an MRI scan acquired using the new sequence discussed above (left). The subject was a 22-year-old male. In FIG. 10, each of the images represents a sample image acquired every two seconds from a series of 108 consecutive scans. As shown in FIG. 10, the difference between the phase contrast images and the images using the new sequence is readily apparent. In particular, the images on the right (generated with a sequence according to the present disclosure) are significantly clearer, allowing physiological structures to be easily identified than in the images on the left (generated with a conventional sequence as in the prior art). Moreover, when images from all 108 scans are played consecutively as a movie, the pulsating for blood flow and CSF can be observed clearly.

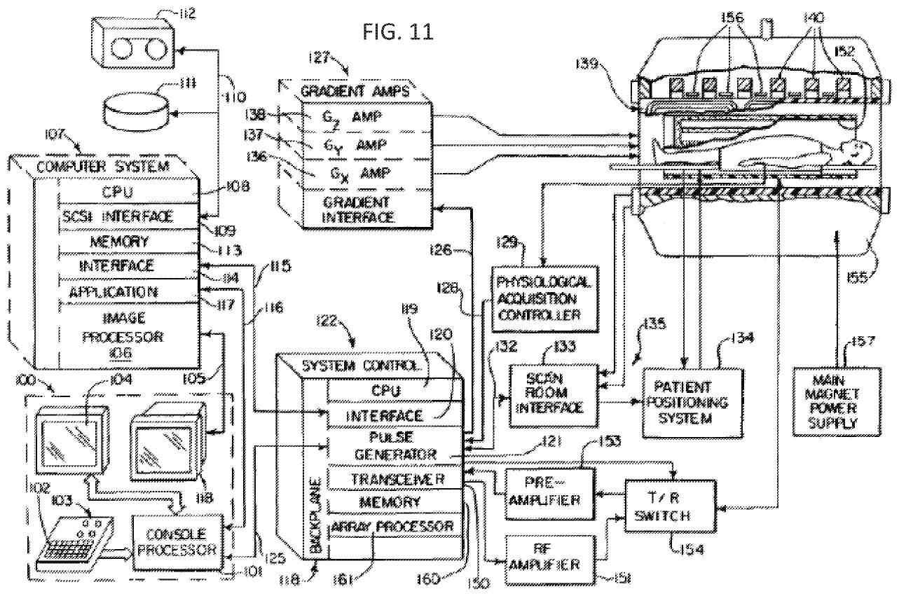

Referring now to FIG. 11, there is shown the major components of an exemplary MRI system which can be used to carry out the methods of the various embodiments. It should be noted that the methods of the various can also be carried out using other MRI systems, including systems with more or less components than shown in FIG. 11.

The operation of the system of FIG. 11 is controlled from an operator console 100 which includes a console processor 101 that scans a keyboard 102 and receives inputs from a human operator through a control panel 103 and a plasma display/touch screen 104. The console processor101 communicates through a communications link 116 with an applications interface module 117 in a separate computer system 107. Through the keyboard 102 and controls 103, an operator controls the production and display of images by an image processor 106 in the computer system 107, which connects directly to a video display 118 on the console 100 through a video cable 105.

The computer system 107 is formed about a backplane bus which conforms with the VME standards, and it includes a number of modules which communicate with each other through this backplane. In addition to the application interface 117 and the image processor 106, these include a CPU module 108 that controls the VME backplane, and an SCSI interface module 109 that connects the computer system 107 through a bus 110 to a set of peripheral devices, including disk storagel11 and tape drive 112. The computer system 107 also includes a memory module 113, known in the art as a frame buffer for storing image data arrays, and a serial interface module 114 that links the computer system 107 through a high speed serial link 115 to a system interface module 120 located in a separate system control cabinet 122.

The system control 122 includes a series of modules which are connected together by a common backplane 118. The backplane 118 is comprised of a number of bus structures, including a bus structure which is controlled by a CPU module 119. The serial interface module 120 connects this backplane 118 to the high speed serial link 115, and pulse generator module 121 connects the backplane 118 to the operator console 100 through a serial link 125. It is through this link 125 that the system control 122 receives commands from the operator which indicate the scan sequence that is to be performed.

The pulse generator module 121 operates the system components to carry out the desired scan sequence. It produces data which indicates the timing, strength and shape of the RF pulses which are to be produced, and the timing of and length of the data acquisition window. The pulse generator module 121 also connects through serial link 126 to a set of gradient amplifiers 127, and it conveys data thereto which indicates the timing and shape of the gradient pulses that are to be produced during the scan. The pulse generator module 121 also receives patient data through a serial link 128 from a physiological acquisition controller 129. The physiological acquisition control 129 can receive a signal from a number of different sensors connected to the patient. For example, it may receive ECG signals from electrodes or respiratory signals from a bellows and produce pulses for the pulse generator module 121 that synchronizes the scan with the patient's cardiac cycle or respiratory cycle. And finally, the pulse generator module 121 connects through a serial link 132 to scan room interface circuit 133 which receives signals at inputs 135 from various sensors associated with the position and condition of the patient and the magnet system. It is also through the scan room interface circuit 133 that a patient positioning system134 receives commands which move the patient cradle and transport the patient to the desired position for the scan.

The gradient waveforms produced by the pulse generator module 121 are applied to a gradient amplifier system 127 comprised of Gₓ, G_{y}, and G_{z} amplifiers 136, 137 and 138, respectively. Each amplifier 136, 137 and 138 is utilized to excite a corresponding gradient coil in an assembly generally designated 139. The gradient coil assembly 139 forms part of a magnet assembly 155 which includes a polarizing magnet 140 that produces a 1.5 Tesla polarizing field that extends horizontally through a bore. The gradient coils 139 encircle the bore, and when energized, they generate magnetic fields in the same direction as the main polarizing magnetic field, but with gradients Gₓ, G_{y} and G_{z} directed in the orthogonal x-, y- and z-axis directions of a Cartesian coordinate system. That is, if the magnetic field generated by the main magnet 140 is directed in the z direction and is termed BO, and the total magnetic field in the z direction is referred to as B_{z}, then Gₓ∂B_{z}/∂x, G_{y}==∂B_{z}/∂y and G_{z}=∂B_{z}/∂z, and the magnetic field at any point (x,y,z) in the bore of the magnet assembly 141 is given by B(x,y,z)=Bₒ+Gₓx+G_{y}yG_{z}z. The gradient magnetic fields are utilized to encode spatial information into the MRI signals emanating from the patient being scanned. Because the gradient fields are switched at a very high speed when an EPI sequence is used to practice the preferred embodiment of the invention, local gradient coils are employed in place of the whole-body gradient coils 139. These local gradient coils are designed for the head and are in close proximity thereto. This enables the inductance of the local gradient coils to be reduced and the gradient switching rates increased as required for the EPI pulse sequence. For a description of these local gradient coils which is incorporated herein by reference, see U.S. Pat. No. 5,372,137 issued on Dec. 13, 1994 and entitled “MRI Local Coil For Brain Imaging”.

Located within the bore 142 is a circular cylindrical whole-body RF coil 152. However, other configurations can be more limited. This coil 152 produces a circularly polarized RF field in response to RF pulses provided by a transceiver module 150 in the system control cabinet 122. These pulses are amplified by an RF amplifier 151 and coupled to the RF coil 152 by a transmit/receive switch 154 which forms an integral part of the RF coil assembly. Waveforms and control signals are provided by the pulse generator module 121 and utilized by the transceiver module 150 for RF carrier modulation and mode control. The resulting MRI signals radiated by the excited nuclei in the patient may be sensed by the same RF coil 152 and coupled through the transmit/receive switch 154 to a preamplifier 153. The amplified MRI signals are demodulated, filtered, and digitized in the receiver section of the transceiver150.

The transmit/receive switch 154 is controlled by a signal from the pulse generator module 121 to electrically connect the RF amplifier 151 to the coil 152 during the transmit mode and to connect the preamplifier 153 during the receive mode. The transmit/receive switch 154 also enables a separate local RF head coil to be used in the transmit and receive mode to improve the signal-to-noise ratio of the received MRI signals. With currently available MRI systems such a local RF coil is preferred in order to detect small variations in MRI signal. Reference is made to the above cited U.S. Pat. No. 5,372,137 for a description of the preferred local RF coil.

In addition to supporting the polarizing magnet 140 and the gradient coils 139 and RF coil 152, the main magnet assembly 141 also supports a set of shim coils 156 associated with the main magnet 140 and used to correct inhomogeneities in the polarizing magnet field. The main power supply 157 is utilized to bring the polarizing field produced by the superconductive main magnet 140 to the proper operating strength and is then removed.

The MRI signals picked up by the RF coil are digitized by the transceiver module 150 and transferred to a memory module 160 which is also part of the system control 122. When the scan is completed and an entire array of data has been acquired in the memory modules 160, an array processor 161 operates to Fourier transform the data into an array of image data. This image data is conveyed through the serial link 115 to the computer system 107 where it is stored in the disk memory 111. In response to commands received from the operator console 100, this image data may be archived on the tape drive 112, or it may be further processed by the image processor 106 and conveyed to the operator console 100 and presented on the video display 118 as will be described in more detail hereinafter.

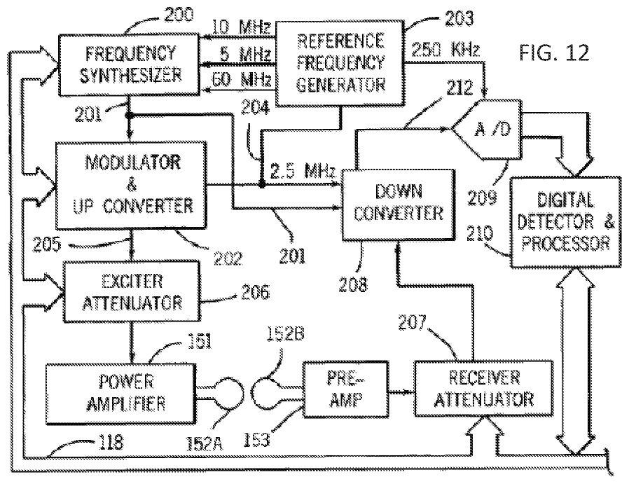

Referring particularly to FIGS. 11 and 12, the transceiver 150 includes components which produce the RF excitation field B1 through power amplifier 151 at a coil 152A and components which receive the resulting MRI signal induced in a coil 152B. As indicated above, the coils 152A and B may be a single whole-body coil, but the best results are achieved with a single local RF coil specially designed for the head. The base, or carrier, frequency of the RF excitation field is produced under control of a frequency synthesizer 200 which receives a set of digital signals (CF) through the backplane 118 from the CPU module 119 and pulse generator module 121. These digital signals indicate the frequency and phase of the RF carrier signal which is produced at an output 201. The commanded RF carrier is applied to a modulator and up converter 202 where its amplitude is modulated in response to a signal R(t) also received through the backplane 118 from the pulse generator module 121. The signal R(t) defines the envelope, and therefore the bandwidth, of the RF excitation pulse to be produced. It is produced in the module 121 by sequentially reading out a series of stored digital values that represent the; desired envelope. These stored digital values may, in turn, be changed from the operator console 100 to enable any desired RF pulse envelope to be produced. The modulator and up converter 202 produces an RF pulse at the desired Larmor frequency at an output 205. The magnitude of the RF excitation pulse output through line 205 is attenuated by an exciter attenuator circuit 206 which receives a digital command, TA, from the backplane 118. The attenuated RF excitation pulses are applied to the power amplifier 151 that drives the RF coil 152A. For a more detailed description of this portion of the transceiver 122, reference is made to U.S. Pat. No. 4,952,877 which is incorporated herein by reference.

Referring still to FIGS. 11 and 12 the MRI signal produced by the subject is picked up by the receiver coil 152B and applied through the preamplifier 153 to the input of a receiver attenuator 207. The receiver attenuator 207 further amplifies the MRI signal and this is attenuated by an amount determined by a digital attenuation signal (RA) received from the backplane 118. The receive attenuator 207 is also turned on and off by a signal from the pulse generator module 121 such that it is not overloaded during RF excitation. The received MRI signal is at or around the Larmor frequency, which in the preferred embodiment is around 63.86 MHz for 1.5 Tesla. This high frequency signal is down converted in a two-step process by a down converter 208 which first mixes the MRI signal with the carrier signal on line 201 and then mixes the resulting difference signal with the 2.5 MHz reference signal on line 204. The resulting down converted MRI signal on line 212 has a maximum bandwidth of 125 kHz and it is centered at a frequency of 187.5 kHz. The down converted MRI signal is applied to the input of an analog-to-digital (A/D) converter 209 which samples and digitizes the analog signal at a rate of 250 kHz. The output of the A/D converter 209 is applied to a digital detector and signal processor 210 which produce 16-bit in-phase (1) values and 16-bit quadrature (Q) values corresponding to the received digital signal. The resulting stream of digitized I and Q values of the received MRI signal is output through backplane 118 to the memory module 160 where they are employed to reconstruct an image.

To preserve the phase information contained in the received MRI signal, both the modulator and up converter 202 in the exciter section and the down converter 208 in the receiver section are operated with common signals. More particularly, the carrier signal at the output 201 of the frequency synthesizer 200 and the 2.5 MHz reference signal at the output 204 of the reference frequency generator 203 are employed in both frequency conversion processes. Phase consistency is thus maintained and phase changes in the detected MRI signal accurately indicate phase changes produced by the excited spins. The 2.5 MHz reference signal as well as 5, 10 and 60 MHz reference signals are produced by the reference frequency generator 203 from a common 20 MHz master clock signal. The latter three reference signals are employed by the frequency synthesizer 200 to produce the carrier signal on output 201. For a more detailed description of the receiver, reference is made to U.S. Pat. No. 4,992,736 which is incorporated herein by reference.

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments. Rather, the scope of the invention should be defined in accordance with the following claims and their equivalents.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

What is claimed is:

1. A method, comprising:
obtaining a plurality of magnetic resonance imaging (MRI) scans of a cerebral vessel, wherein each of the plurality of MRI scans comprises a modified True FISP sequence, wherein the modified True FISP sequence comprises modified True FISP parameters related to a resolution and a speed of imaging, the modified True FISP parameters yielding a scan image with identifiable physiological structures;
outputting scan images based on the plurality of MRI scans;
based on the scan images of each of the plurality of MRI scans, identifying a flow within the cerebral vessel;
identifying pulsatile features of the identified flow within the cerebral vessel;
determining whether the identified pulsatile features are related to a disease state; and
outputting an indication of the disease state based on determining whether the identified pulsatile features are related to a disease state.

2. The method of claim 1, wherein the modified True FISP parameters comprises field of view and resolution matrix.

3. The method of claim 1, wherein the plurality of MRI scans is obtained consecutively over a set period of time, with a set interval between each scan of the plurality of MRI scans.

4. The method of claim 1, wherein visualizing the flow within the cerebral vessel comprises generating a 3-D representation of fluid moving through the cerebral vessel over a set period of time.

5. The method of claim 1, wherein visualizing the flow within the cerebral vessel comprises assembling the plurality of MRI scans into a video file.

6. The method of claim 1, wherein the identified pulsatile features comprises at least one of: flow pressure, flow pulsatility, and flow velocity.

7. The method of claim 1, wherein determining whether the identified pulsatile features are related to a disease state comprises:
comparing the identified pulsatile features to corresponding threshold values.

8. The method of claim 1, wherein the disease state comprises dementia, stroke, hydrocephalus, arteriovenous malformations, aneurysms, renal artery stenosis, development of atherosclerotic disease, or peripheral vascular disease.

9. A system, comprising:
an magnetic resonance imaging (MRI) system;
a computing device communicatively coupled to the MRI system; and
a memory having stored thereon a computer program for operating the computing device, the computer program comprising a plurality of code sections for causing the computing device to perform steps comprising:
controlling the MRI system so as to acquire a plurality of consecutive MRI scans of a cerebral vessel using a modified True FISP sequence, wherein each of the plurality of consecutive MRI scans yields a scan image;
outputting scan images of each of the plurality of MRI scans;
identifying a flow within the cerebral vessel, based on the scan images of each of the plurality of consecutive MRI scans;
identifying a feature of the flow related to a disease state; and
outputting an indication of the identified feature to a display.

10. The system of claim 9, wherein identifying the flow within the cerebral vessel comprises assembling the plurality of consecutive MRI scans into a video file.

11. The system of claim 9, wherein the identified feature of the flow comprises at least one of: flow pressure, flow pulsatility, or flow velocity.

12. The system of claim 9, wherein the modified True FISP sequence comprises a modified field of view and resolution matrix.

13. The system of claim 9, further comprising assessing a disease state of the cerebral vessel based on the identified feature of the flow.

14. The system of claim 13, wherein assessing a disease state comprises comparing the identified feature to a threshold value associated with the identified feature.

15. The system of claim 9, wherein the disease state comprises dementia, stroke, hydrocephalus, arteriovenous malformations, aneurysms, renal artery stenosis, development of atherosclerotic disease, or peripheral vascular disease.
